SEQUENCE LISTING (4 sequences)

SEQ ID NO: 1 - protein, 309 aa, Artificial sequence
  RBD-VSVG fusion protein
MKCLLYLAFLFIGVNCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGDTGLSKNPIELVEGWFSSWKSSIASFFFIIGLIIGLFLVLRVGIHLCIKLKHTKKRQIYTDIEMNRLGK

SEQ ID NO: 2 - protein, 16 aa, Artificial sequence
  Signal peptide
MKCLLYLAFLFIGVNC

SEQ ID NO: 3 - DNA, 28 bp, Artificial sequence
  Primer for RBD
atgtttcctaatattacaaacttgtgcc

SEQ ID NO: 4 - DNA, 25 bp, Artificial sequence
  Primer for RBD
ttatgctggtgcatgtagaagttca

(19) United States
(12) Patent Application Publication
(10) Pub. No.: US 2022/0348954 A1
(43) Pub. Date: Nov. 3, 2022

(54) TARGETED EXOSOME BASED ON RBD REGION OF SARS-COV-2 S PROTEIN AND PREPARATION METHOD THEREOF

(71) Applicant: SOOCHOW UNIVERSITY, Suzhou (CN)

(72) Inventors: Sidong XIONG, Suzhou (CN); Yuxuan FU, Suzhou (CN)

(21) Appl. No.: 17/631,439

(22) PCT Filed: Jul. 8, 2021
(86) PCT No.: PCT/CN2021/105245

§ 371 Date: Jan. 28, 2022

(30) Foreign Application Priority Data
Oct. 14, 2020 (CN) 202011097744.1

(51) Int. Cl. C12N 15/85; C07K 14/005; A61P 31/14; A61K 47/46; A61K 47/42

(52) U.S. Cl. CPC C12N 15/85; C07K 14/005; A61P 31/14; A61K 47/46; A61K 47/42; C12N 2760/20222; C12N 2509/10; C12N 2800/107; C12N 2770/20022; C07K 2319/02

(57) ABSTRACT

The present invention discloses a targeted exosome based on the RBD region of SARS-CoV-2 S protein and a preparation method thereof. An RBD-VSVG fusion protein is expressed on the targeted exosome of the present invention, and the RBD-VSVG fusion protein is obtained by replacing the extracellular region of VSVG with the RBD of the SARS-CoV-2 S protein. In the present invention, a targeted exosome capable of efficiently and tissue-specifically delivering a potential anti-SARS-CoV-2 medicine is constructed. The targeted exosome is used to encapsulate SARS-CoV-2 siRNA, to specifically inhibit the virus replication in tissues and organs. In a mouse animal model, tail vein injection of exosome encapsulated SARS-CoV-2 siRNA significantly inhibits virus replication in mouse lung tissue and alleviates symptoms such as pneumonia caused by virus infection.

### FIELD OF THE INVENTION

The present invention relates to the technical field of biomedicines, and more particularly to a targeted exosome based on the RBD region of SARS-CoV-2 S protein and a preparation method thereof.

### DESCRIPTION OF THE RELATED ART

Severe Acute Respiratory Syndrome Coronavirus Type II (SARS-CoV-2) is the cause of Coronavirus Disease 2019 (COVID-19), which has a rising mortality and has become a major global public health issue. So far, no specific medicine or vaccine has been officially approved. Structural analysis and pathological observation confirm that SARS-CoV-2 virus enters tissues and organs by binding to angiotensin-converting enzyme 2 (ACE-2) on the host cells, and the entry of that virus into cells depends on the receptor binding domain (RBD) of the SARS-CoV-2 spike protein (S) which specifically recognizes ACE2. Currently, it is believed that blocking the binding of RBD to ACE2 is a main potential strategy in the development of vaccines, neutralizing antibodies and small molecule drugs against COVID-19.

Exosomes are natural transport nanovesicles (approximately 30-100 nm) secreted by a variety of cells. It is currently known that exosome can deliver specific functional biomolecules (such as a nucleic acid, including a plasmid DNA and a small interfering RNA, an antibody, and a small molecule drug) to recipient cells or tissues and organs to exert their ability to treat specific diseases. However, studies have shown that most of the intravenously injected exosomes are absorbed and metabolized by the liver. Therefore, the exosome needs to be modified for targeted treatment to deliver an exogenous therapeutic medicine to specific cells or tissues in the body. Till now, there is no targeted carrier for specific delivering a medicine for the treatment of COVID-19. In the present invention, the exosome is modified to target SARS-CoV-2 specific tissues and organs, so as to provide a targeted carrier for the delivery of a related specific anti-viral medicine, thereby achieving the precise treatment for SARS-CoV-2 infection.

### SUMMARY OF THE INVENTION

In order to solve the above-mentioned problems, the present invention provides a targeted exosome based on the RBD region of SARS-CoV-2 S protein, which can efficiently and tissue-specifically deliver a potential anti-SARS-CoV-2 medicine.

A first object of the present invention is to provide a targeted exosome based on the RBD region of the SARS-CoV-2 S protein. A RBD-VSVG fusion protein is expressed on the targeted exosome, and the RBD-VSVG fusion protein is obtained by replacing the extracellular region of VSVG (glycoprotein G of vesicular stomatitis virus) with the RBD (receptor binding domain) of the SARS-CoV-2 (Severe Acute Respiratory Syndrome Coronavirus Type II) S protein (spike protein).

Preferably, the amino acid sequence of the RBD-VSVG fusion protein is as shown in SEQ ID NO:1.

Preferably, the N-terminus of the RBD-VSVG fusion protein is provided with a signal peptide.

Preferably, the amino acid sequence of the signal peptide is as shown in SEQ ID NO:2.

A second object of the present invention is to provide a method for preparing the targeted exosome, comprising the steps of:

S1: obtaining a RBD fragment by PCR amplification using a sample cDNA containing SARS-CoV-2 as a template;

S2: in vitro synthesizing a full-length gene fragment of a transmembrane region and an intracellular region of VSVG;

S3: ligating the RBD fragment of step S1 and the full-length gene fragment of the transmembrane region and the intracellular region of VSVG of step S2 to a vector, to obtain an expression vector;

S4: transferring the expression vector of step S3 into host cells, culturing the host cells and collecting the cell culture supernatant, and isolating the targeted exosome.

Preferably, the host cell is a 293T cell or a dendritic cell.

Preferably, the vector is a pCMV vector.

Preferably, the step of isolating the targeted exosome further includes the steps of: centrifuging the cell culture supernatant at 8,000-15,000 g for 20-40 min and collecting the supernatant, filtering the supernatant through a micron-level filter membrane, ultracentrifuging at 80,000-120,000 g for 60-80 min and collecting the precipitation; resuspending the precipitation in a buffer, ultracentrifuging at 80,000-120,000 g for 60-80 min, and removing the supernatant to obtain the exosome.

A third object of the present invention is to provide use of the targeted exosome in the preparation of a medicine for the targeted treatment of COVID-19. The use comprises transferring a specific anti-SARS-CoV-2 functional biomolecule into the targeted exosome, to obtain the medicine for the targeted treatment of COVID-19.

Preferably, the functional biomolecule is a small interfering RNA, an antibody or a small molecule drug.

The beneficial effects of the present invention are as follows.

In the present invention, a targeted exosome capable of efficiently and tissue-specifically delivering a potential anti-SARS-CoV-2 medicine is provided. The targeted exosome is used to encapsulate SARS-CoV-2 siRNA, to specifically inhibit virus replication in tissues and organs.

In a mouse animal model, tail vein injection of exosome encapsulated SARS-CoV-2 siRNA can significantly inhibit virus replication in mouse lung tissue and alleviate symptoms such as pneumonia caused by virus infection.

### BRIEF DESCRIPTION OF THE DRAWINGS

FIG. 1 schematically shows cells with glycoprotein G of vesicular stomatitis virus (VSVG) replaced by the RBD region of SARS-CoV-2 S protein;

FIG. 2 is a TEM image of normal cell exosome and RBD-labeled exosome;

FIG. 3 shows nanoparticle analysis of normal cell exosome and RBD-labeled exosome;

FIG. 4 shows the immunoprecipitation results of normal cell exosome and RBD-labeled exosome;

FIG. 5 shows the targeted enrichment of normal cell exosome and RBD-labeled exosome;

FIG. 6 shows the effect of normal cell exosome and RBD-labeled exosome carrying an active antiviral medicine on inhibiting the virus.

### DETAILED DESCRIPTION OF THE PREFERRED EMBODIMENTS

The present invention will be further described below in combination with specific examples, so that those skilled in the art can better understand and implement the present invention, but the examples provided herein are not intended to limit the present invention.

### Example 1

1. The primers for RBD (F: 5′-ATGTTTCCTAATATTACAAACTTGTGCC-3′, SEQ ID NO: 3; R: 5′-TTATGCTGGTGCATGTAGAAGTTCA-3′, SEQ ID NO: 4) were designed. Sample cDNA of a throat swab sample from a COVID-19 patient was used as a template, and amplified by using the TaKaRa PCR kit to obtain a complete RBD fragment. After 2% agarose electrophoresis, the Axygen gel extraction kit were used to purify the DNA product. The full-length DNA of the transmembrane region and the intracellular region of VSVG was synthesized in vitro and inserted into the pCMV vector by T4 ligase to construct the pCMV-VSVG vector, which was sequenced for sequence verification.

2. The PCR product, RBD gene, purified and recovered in step 1 was ligated to pCMV-VSVG vector by T4 ligase, and reacted overnight at 16° C. to construct the pCMV-RBD-VSVG vector, which was sequenced for sequence verification. Cells were transformed with the vector, and plated. Subsequently, a single clone was picked up and expanded. The pCMV-RBD-VSVG plasmid vector was extracted by Axygen plasmid extraction kit.

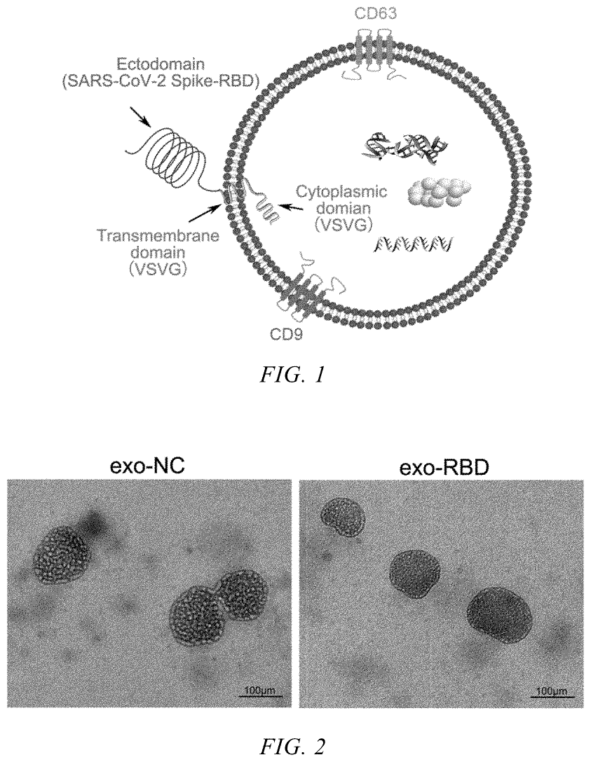

FIG. 1 shows that the extracellular region of glycoprotein G of the vesicular stomatitis virus (VSVG) is replaced with the RBD region of SARS-CoV-2 S protein to form a fusion vector of RBD and VSVG. After being transfected into a cell for expression, the obtained exosome expresses RBD-VSVG fusion protein, in which the extracellular region is the RBD protein, and the transmembrane and intracellular regions are the transmembrane and intracellular regions of the VSVG protein. CD9 and CD63 are exosome-specific marker proteins.

3. 293T cells were plated (100 mm dish) with a density of about 60-70%. The cells were transfected with the pCMV-RBD-VSVG recombinant vector in step 2 by PEI (the weight ratio of plasmid to PEI is 1:3). After 6 hrs, the culture medium was replaced with an exosome-free medium. The cell culture supernatant was collected after the culture was continued for 48 hrs.

4. The collected supernatant was centrifuged at 10,000 g and 4° C. for 30 min. The cell debris was removed. Then, the supernatant after centrifugation was filtered through a 0.22 micron filter membrane, and centrifuged for 70 min at 100,000 g and 4° C. in an ultracentrifuge (Beckman, Germany). The supernatant was carefully removed, and an appropriate amount of PBS was added. The exosome precipitation was suspended and mixed well by pipetting, then centrifuged at 100,000 g and 4° C. for 70 min. The supernatant was removed. The precipitation was resuspended in an appropriate amount of PBS to obtain the targeted exosome. The size of the exosome was detected by electron microscopy and a nano particle size analyzer.

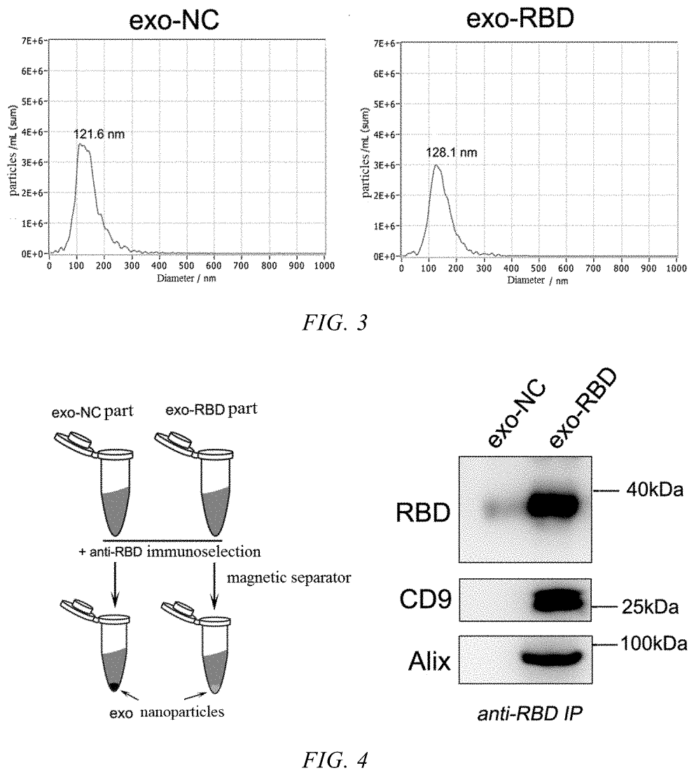

FIGS. 2 and 3 compare the extracted exosome with the exosome secreted by normal cells (exo-NC) by transmission electron microscopy (TEM) and nanoparticle analysis (NTA), respectively. RBD-labeled exosome (exo-RBD) has no significant difference in size, indicating that the RBD labelling does not affect the normal physical morphology and characteristics of the exosome.

By the exosome immunoprecipitation technique, normal cell exosome (exo-NC) and RBD-labeled exosome (exo-RBD) were respectively incubated with magnetic beads coupled with the RBD antibody, and then separated by a magnetic separator. FIG. 4 shows that the RBD protein is expressed on the outer membrane of the exosome, as detected by western blot.

5. The siRNA for SARS-CoV-2 was designed and synthesized. The specific interfering RNA (siRNA) against SARS-CoV-2 genome was electroporated into the obtained targeted exosome by an electroporator (Bio-Rad). The targeted exosome was allowed to encapsulate siRNA for delivery at a weight ratio of 1:1 of exosome: siRNA. The resultant material was centrifuged at 100,000 g and 4° C. for 70 min in an ultracentrifuge (Beckman, Germany). After excess siRNA was removed, the exosome precipitation was suspended in PBS.

6. The humanized ACE2 (SARS-CoV-2 specific receptor) mice were used as a model. The targeted exosome was injected through tail vein with 150 ug/mouse, to achieve targeted delivery of siRNA to SARS-CoV-2 tropic tissues and organs and replication of infected viruses.

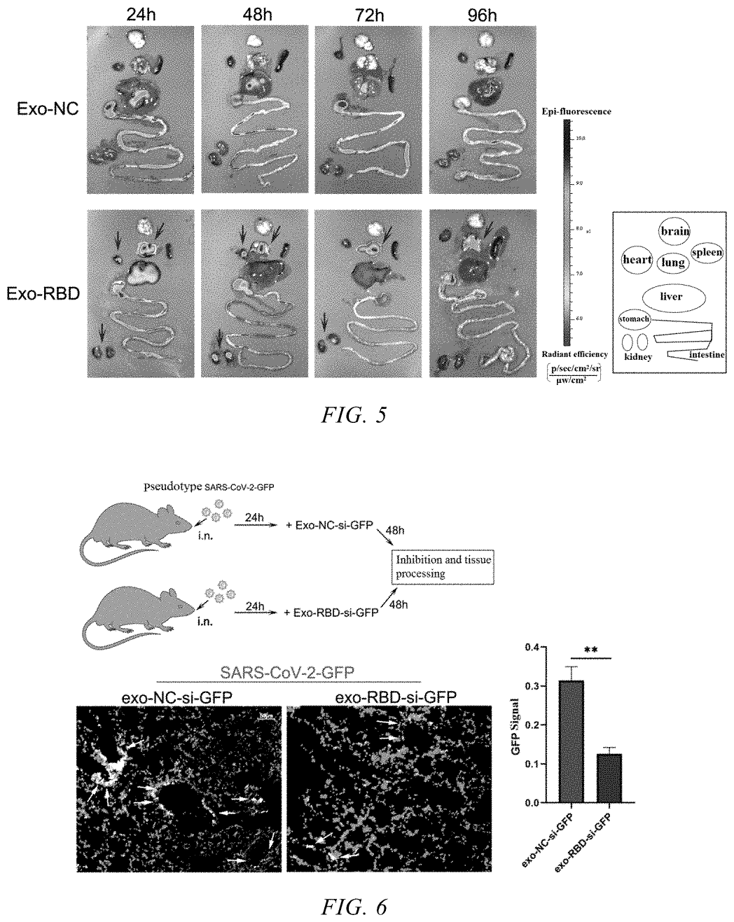

The normal cell exosome (exo-NC) and RBD-labeled exosome (exo-RBD) were respectively labeled with DiD lipophilic dye for fluorescence, and injected into humanized ACE2 mice through the tail vein. Continuous observation was carried out for 96 hrs with a time interval of 24 hrs. The results in FIG. 5 show that exo-RBD can be significantly enriched in mouse lung tissue, heart and kidney tissue for a period of not less than 96 hrs. The normal exosome exo-NC which is not labeled with RBD protein is not enriched in the above-mentioned tissues.

Humanized ACE2 mice were infected with the SARS-CoV-2 pseudovirus with green fluorescent protein GFP through the nasal drip route. After 24 hrs, normal cell exosome (exo-NC) carrying GFP siRNA and RBD-labeled exosome (exo-RBD) carrying GFP siRNA were respectively injected via the tail vein. After 48 hrs, the fluorescence intensity of GFP in mouse lung tissue was detected by tissue immunofluorescence. The results in FIG. 6 show that the RBD-labeled exosome carrying GFP siRNA can significantly inhibit the expression of GFP of SARS-CoV-2 pseudovirus, indicating that RBD-labeled exosome can be used as an effective carrier, carrying an active antiviral medicine to target SARS-CoV-2 tropic tissues (lungs, etc.), to inhibit the pathogenicity of the virus.

The above-mentioned embodiments are merely preferred embodiments provided for the purpose of fully illustrating the present invention, and the protection scope of the present invention is not limited thereto. Equivalent substitutions or alterations made by those skilled in the art on the basis of the present invention fall into the protection scope of the present invention as defined by the claims.

1. A targeted exosome based on the RBD region of SARS-CoV-2 S protein, wherein a RBD-VSVG fusion protein is expressed on the targeted exosome, which is obtained by replacing the extracellular region of VSVG with the RBD of the SARS-CoV-2 S protein.

2. The targeted exosome according to claim 1, wherein an amino acid sequence of the RBD-VSVG fusion protein is as shown in SEQ ID NO:1.

3. The targeted exosome according to claim 2, wherein the N-terminus of the RBD-VSVG fusion protein is provided with a signal peptide.

4. The targeted exosome according to claim 3, wherein an amino acid sequence of the signal peptide is as shown in SEQ ID NO:2.

5. A method for preparing the targeted exosome according to claim 1, comprising steps of:
S1: obtaining a RBD fragment by PCR amplification using a sample cDNA containing SARS-CoV-2 as a template;
S2: in vitro synthesizing a full-length gene fragment of a transmembrane region and an intracellular region of VSVG;
S3: ligating the RBD fragment of step S1 and the full-length gene fragment of the transmembrane region and the intracellular region of VSVG of step S2 to a vector, to obtain an expression vector; and
S4: transferring the expression vector of step S3 into host cells, culturing the host cells and collecting the cell culture supernatant, and isolating the targeted exosome.

6. The method according to claim 5, wherein the host cell is a 293T cell or a dendritic cell.

7. The method according to claim 5, wherein the vector is a pCMV vector.

8. The method according to claim 5, wherein isolating the targeted exosome comprises steps of: centrifuging the cell culture supernatant at 8,000-15,000 g for 20-40 min and collecting the supernatant, filtering the supernatant through a micron-level filter membrane, ultracentrifuging at 80,000-120,000 g for 60-80 min and collecting the precipitation; resuspending the precipitation in a buffer, ultracentrifuging at 80,000-120,000 g for 60-80 min, and removing the supernatant to obtain the exosome.

9. Use of the targeted exosome according to claim 1 in the preparation of a medicine for the targeted treatment of COVID-19, comprising transferring a specific anti-SARS-CoV-2 functional biomolecule into the targeted exosome, to obtain a medicine for the targeted treatment of COVID-19.

10. The use according to claim 9, wherein the functional biomolecule is a small interfering RNA, an antibody or a small molecule drug.
